(19) United States
(12) Patent Application Publication
(10) Pub. No.: US 2009/0294315 A1
(43) Pub. Date: Dec. 3, 2009

(54) Disposal and Packaging Device

(76) Inventor: Martha Selene Silcott, London (GB)

Correspondence Address: JEFFREY DEAN LINDSAY, 20 DIANE LANE, APPLETON, WI, 54915, US

(21) Appl. No.: 12/159,920

(22) PCT Filed: Dec. 22, 2006
(86) PCT No.: PCT/EP06/70180

§ 371 Date: Jul. 2, 2008

(30) Foreign Application Priority Data
Jan. 3, 2006 (GB) 0600024.4
Jun. 9, 2006 (GB) 0611398.9
Oct. 7, 2006 (GB) 0619909.7

(51) Int. Cl. A61B 19/02; B65D 83/00

(52) U.S. Cl. 206/440; 221/312 C

(57) ABSTRACT

A disposal device for the receipt of an item is disclosed. The disposal device comprises a container substantially impervious to liquid having a mouth formed therein through which a soiled item may be received by the container. In use, the mouth is arranged to be capable of manipulation by a single hand of a user between a mouth open position and a mouth closed position. The disposal device may also be configured to act as primary packaging for the item before use (and soiling) thereof or for storage of the item.

### CROSS REFERENCE AND PRIORITY

This application filed under 35 USC 371, is cross-referenced with and claims priority from International Patent Application PCT/EP2006/070180 filed on Dec. 22, 2006, and published as Publication No. WO2007/077172 A1 on Jul. 12, 2007, under PCT article 21(2), which in turn claims priority of GB 0600024.2, filed on Jan. 3, 2006, GB 0611398.9, filed on Jun. 9, 2006, and GB 0619908.7, filed on Oct. 7, 2006, all of which are hereby incorporated by reference in their entireties for all purposes.

### FIELD OF THE INVENTION

The present invention relates to a disposal device suitable for use in the disposal of an item. The disposal device herein may be configured for use as packaging for the item and/or storage of the item.

### BACKGROUND

There are many situations in every day life where manual disposal of soiled items is required which may be inconvenient, unhygienic or problematic in some other way, for example, by disposal in an environmentally unfriendly way. Such difficulties may particularly arise in relation to the disposal of sanitary products or of products contaminated with bodily fluid, for example, in a medical environment. Similarly, it may be difficult to dispose of products that have been exposed to chemical matter in, for example, the chemical or food industries. Alternatively, it may be difficult to receive forensic or medical items within a device without “contaminating” the forensic or medical information carried thereby.

When manually disposing of a soiled item, it is possible that a person may be holding the item whilst attempting to manipulate a disposal device such as a bin and so have only one free hand available for assisting in the disposal process, for example, by opening the bin. Alternatively, it may be that the person does not want to touch the soiled item with more than one hand, to prevent contamination of other articles or if, for example, wearing only one glove. Further problems may arise if it is necessary to carry the soiled item, for example, in the absence of bins.

One particular problem arises in the disposal of soiled tampons, tampon applicators or sanitary towel products where the woman will typically remove the soiled tampon, tampon applicator or sanitary towel from her vaginal region with one hand, thereby leaving only one free hand to assist further in the disposal process by for example, opening a receptacle (e.g. a bin or bag) for the receipt of the soiled item.

It is known to provide plastic bags for such purposes where two handles of the bag have to be tied together to close the bag to prevent the soiled tampon or sanitary towel from escaping. Such prior art plastic bags are, however, difficult to extract and open with one free hand to ensure effective receipt of the soiled item without in particular, soiling or contaminating the hands or clothing of the user during the bag opening and/or soiled item receiving process. Thus, there is a need for improved disposal devices that are convenient and hygienic for use by the user. There is also a need for a disposal device that may be manipulated with one hand for receipt of an item.

There is also a need for disposal devices that may be hygienically sealed in an inexpensive and convenient manner.

A further aspect of the present invention provides a dispenser for dispensing of a disposal device herein.

### SUMMARY

According to a first aspect of the present invention there is provided a disposal device for the receipt of an item, the disposal device comprising a container substantially impervious to liquid having a mouth formed therein through which a soiled item may be received by the container, wherein in use, the mouth is arranged to be capable of manipulation by a single hand of a user between a mouth open position and a mouth closed position.

The term “disposal device” as used herein refers to a device that is suitable for the receipt of an item such as an item to be disposed. In some embodiments, that item is a soiled item is for example, a soiled tampon, sanitary napkin, medical item or condom, which is received by the device for disposal thereof (e.g., in a waste bin or incinerator). In other embodiments, that item is for example, a forensic or medical item that may be soiled in the sense of not being sterile, but rather is soiled (or contaminated) with material containing information of a forensic or medical quality (e.g. DNA information), and which is received by the device for stored receipt thereof e.g. for later inspection and analysis. Thus, in particular examples of these other embodiments the disposal device may function as a “sample bag” for disposed receipt of forensic or medical items. In other embodiments, that item is unsoiled (e.g. in the sense of not having being used or in the case of for example, medical products, being sterile) and the device is primarily used for packaging and/or storage of the item. Thus in some embodiments, a plethora of items such as food stuffs, nuts and bolts and haberdashery items, may also be stored/carried in the device and after use, potentially subsequently disposed of in the device.

A particular area where such a disposal device may be required is in the disposal of soiled sanitary or medical products. Typically, sanitary products such as tampons and sanitary towels are disposed of by either flushing them down the lavatory into the public sewerage system or by disposing of them in sanitary bags or bins which are then passed into the public refuse disposal system. Bins which process the soiled item mechanically before subsequent disposal are also known.

Disposal through the public sewerage system is highly undesirable from an environmental point of view due to the need to process sewage containing the sanitary products which may involve physical separation and in some cases, sanitary products passing beyond the sewage process into open water. Physical separation in the sewage process adds to cost and complexity. Moreover, disposal of sanitary products down the lavatory frequently causes blockages of lavatory systems, leading to inconvenience, expense and potential embarrassment.

Disposal using bins, bags or the like presents other problems. For example, in some cases, when disposing of a soiled sanitary product, it is preferable for the soiled item to come into contact with only one hand of the person, thus allowing the person to use the other hand to manipulate other objects like bin lids, bags or taps. Therefore it is preferable that the person should be able to manipulate the disposal device with one hand while holding the soiled item in the other. This enables the person to prevent contamination of other objects around them that may be touched prior to the person having the opportunity to clean their hands.

When used for the disposal of sanitary products, the disposal device of the present invention may avoid the risk of causing blockage to the lavatory system, and may avoid environmental problems associated with removal of the sanitary product from sewage systems, providing a simple, convenient, discreet and hygienic means of disposal of sanitary products. This may be especially advantageous when using facilities that are available to a number of people, for example, in hotels or in a work place. Cleaning of disposal bins may also be made more convenient and less distasteful as the disposal device of the present invention may be placed in a bin without risk of soiling the bin itself.

In embodiments of the present invention suited for disposal of a soiled item, the soiled item may be one that has come into contact with bodily fluids such as a soiled tampon, sanitary napkin, condom, medical item or forensic item. From a hygiene standpoint, effective receipt (e.g. for disposal or sample storage) of such soiled items is often desirable. From a user standpoint, the device may enable effective receipt of the soiled item without risk of mess or contamination by (or indeed, of) the soil carried by the item.

The disposal device comprises a container that is substantially impervious to liquid. The container may be suitably in the form of a bag, sachet or pouch. Tapered and concertina bag, sachet or pouch forms are also envisaged.

Gusseting may also be provided to the bag, sachet or pouch to enhance the available volume for disposal of the soiled item.

The container may comprise a plastic polymeric material (e.g. polythene) or a treated (e.g. waxed) paper material. The container may be formed from a biodegradable material, which however still functions to provide the necessary liquid imperviousness during soiled item disposal.

The container has a mouth formed therein through which a soiled item may be received by the container. In use, the mouth is arranged to be capable of manipulation by a single hand of a user between a mouth open position and a mouth closed position.

In a preferred embodiment, the device further comprises mouth manipulation means located on an outside wall of the container for manipulating the mouth between the mouth open position and mouth closed position.

Suitably, in use, a person may, using their fingers or thumb, use the mouth manipulation means to pull apart one part of the mouth from the other to open the container. A soiled item that is being held in the other hand may then be deposited into the container through the mouth for receipt within the container.

The mouth manipulation means may comprise at least a first finger-receipt means through, into or against which one or more fingers, or a thumb can be received. The first finger-receipt means may be located on an outside wall of the container and arranged to enable a person to insert at least one finger or thumb through, into or against the finger-receipt means to manipulate open the mouth of the container.

In some embodiments, the mouth manipulation means may further comprise a second finger-receipt means through, into or against which one or more fingers, or a thumb can be inserted. Suitably, the second finger-receipt means is located on an opposite outside wall of the container to enable a person to insert a finger or thumb through, into or against each finger-receipt means and to pull said fingers and/or thumb apart to open the mouth of the container.

The mouth manipulation means may also comprise both a first finger-receipt means located on a first outside wall of the container and a second finger-receipt means locating on a second outside wall of the container. The first and second finger-receipt means may be arranged on either side of (e.g. adjacent to) the mouth.

In some embodiments, the first finger-receipt means is arranged to accommodate the thumb of a user and the second finger-receipt means is arranged to accommodate the index finger of a user. The mouth manipulation action (i.e. mouth opening/closing) is thus, achieved by a thumb and index finger spacing/de-spacing action.

In some embodiments, the mouth manipulation means comprises at least a first loop through which one or more fingers or a thumb can be inserted. The first loop can be located on an outside wall of the container and can be arranged to enable a person, in use, to insert at least one finger or thumb through the loop to manipulate open the mouth of the container.

In some embodiments, the mouth manipulation means further comprises a second loop though which one or more fingers or a thumb can be inserted. Suitably, the second loop is located on an opposite outside wall of the container to enable a person, in use, to insert a finger or thumb through each loop and to pull said fingers and/or thumb apart to open the mouth of the container.

In some embodiments, the mouth manipulation means comprises both a first loop locating on a first outside wall of the container and a second loop locating on a second outside wall of the container. Suitably, the first and second loops are arranged on either side of (e.g. adjacent to) the mouth.

The first loop may be arranged to accommodate the thumb of a user and the second loop is arranged to accommodate the index finger of a user. The mouth manipulation action (i.e. mouth opening/closing) is thus achieved by a thumb and index finger spacing/de-spacing action.

The loop or each loop may be sized and shaped for ready receipt of a user's thumb/finger (e.g. thumb in first loop and index finger in second loop). Suitably, the profile of the finger-receipt each loop is shaped to mirror the flattened/ovular end profile of a user's thumb or finger. That is to say, the loop profile (i.e. the aperture, which the loop defines) in such embodiments may be slightly elongate, for example, defining a generally ovular, parallelogram or rectangular loop profile.

In one aspect, the loop or each loop is arranged such that it may readily adopt both a first “storage” configuration, in which the loop profile is closed (i.e. the loop is flattened) and a second “in use” configuration, in which the loop profile is open (i.e. to define the desired loop profile for finger/thumb receipt). In such an arrangement, the loops may readily be stored flat (e.g. within packaging or in a dispenser pack) but open up to provide the desired loop profile in use.

One or more folds may be provided to the or each loop such that the loop readily adopts (e.g. “pops out” into) the “in use” configuration in the absence of any external pressure on the loop (e.g. as may exist when the device is stored in a dispenser pack or other packaging). Any dispenser device for the disposal device may be adapted to allow at least one loop to “pop out” from a lead disposal device when the dispenser device is in a disposal device dispensing position.

In one aspect, the loops are formed as an integral part of the container (e.g., a continuation of it). In another aspect, the loops seal to an outside wall of the container. The loops may either comprise the same material as that of the container or be formed from a different material.

In one aspect, the mouth of the container is provided with lips that may act in use to guide the receipt of a soiled item through the mouth. The lips may also to an extent act to shield the user's fingers/thumb (particularly those that are within the finger-receipt means) from contamination by the soiled item. The mouth manipulation means (whose primary purpose is for manipulating the mouth between the mouth open position and mouth closed position) are further arranged for manipulating the lips. In particular, the mouth manipulation means enable manipulation of the lips to a lips open (and guiding) position when the mouth is in the mouth open position.

Embodiments are envisaged in which the mouth manipulation means and the lips form part of an integral arrangement. In one particular aspect, a first loop extends from a first lip and a second loop extends from a second lip. As will be understood from the later description, this kind of integral lips/loops arrangement is desirable from a manufacturing simplicity standpoint. Suitably, the lips may be manipulated into the lips open position (and guiding position) before the mouth itself is manipulated open. Thus, in a typical use sequence, the mouth manipulation means are employed to open the lips, and then to open the mouth. Where a reversible seal is provided to the mouth, the latter step may involve unsealing the mouth to open it up.

In some embodiments, the container may be provided with a seal such that a substantially liquid tight seal to the container may be formed. Thus in a typical use process, once the soiled item has been placed in the container, the mouth is closed and the seal applied. In one aspect herein, two boundaries of the mouth can be brought together with the same fingers or thumb to form a seal.

The hygienic seal may be formed by any known method, such as using an adhesive strip on at least one internal boundary (e.g. edge) of the mouth. Alternatively, different materials that mesh together can be provided on each respective internal boundary of the mouth. For example, a hook and loop arrangement may be used where the material on the internal boundary consists of miniature plastic hooks for mouth manipulation with material located on the opposite inner boundary. Alternatively, a tongue and groove arrangement may be used, where a strip having a tongue is provided along one internal boundary of the mouth with a strip having a corresponding groove provided along the other internal boundary of the mouth.

In one aspect, the hygienic seal is arranged to be reversible (i.e. it may be sealed and then subsequently unsealed). In another aspect, the hygienic seal is arranged such that once sealed the seal cannot be broken without damaging the integrity of the container. The latter aspect may be employed in a “tamper proof” disposal device, which is designed for receipt of the soiled item and permanent sealing—any subsequent removal of the item would be by severing open or otherwise damaging the integrity of the container.

In an alternative embodiment, the mouth is held in the open position by the application of manual force. The mouth is biased so that upon release of the force, the mouth moves to the closed position and forms a hygienic seal. In this arrangement, the soiled item may be held in one hand and the disposal device in the other with an applied manual force holding open the mouth. The product may then be deposited into the container through the mouth, at which point the manual pressure will be released whereby the mouth would close.

In this embodiment, the hygienic seal may be formed by rigid strips abutting against each other along their length. The hygienic seal may be vapor tight. To ensure this, the mouth may comprise an adhesive layer disposed so as to bond to another part of the mouth in the closed position. In one related embodiment, both internal boundaries of the mouth of the container may comprise a layer of adhesive.

In one embodiment, the mouth of the container is releasable and resealable, such that the mouth may be closed prior to use and opened and then re-closed during use, wherein the adhesive may remain effective to form a seal on re-closing the mouth. This form of container may be useful while a person is travelling, for example.

The container may be of any shape and size, but generally should be proportioned to receive the items for which it is intended to be used. For sanitary products, it is desired that the container is of such size and shape so as to be able to receive a tampon and/or a sanitary towel and also not too large to be uncomfortable to carry on the person, for example in a hand bag or briefcase. As an example, the container may have a rectangular front side and a rectangular back side which are joined along three of the four boundaries. The fourth boundary generally defines the mouth so that the device is a generally rectangular sachet or pouch. Lip features may also be provided to the mouth of that sachet or pouch.

In one aspect, the container adopts the form of a generally rectangular pouch, which is designed to lie flat in a “storage” configuration. The generally rectangular pouch therefore has two opposing major (i.e. longer) sides and two opposing minor (i.e. shorter) sides. The mouth may be formed at one minor side of the generally rectangular pouch. The generally rectangular pouch may be provided with gusseting at the major sides of the rectangle, which define the length of the pouch. The gusseting may run parallel to the major sides or may be provided at a slight angle (e.g. 10 to 20 degrees) thereto.

In some embodiments, the disposal device is arranged for ease of folding into a folded-up state. When the device is in the folded state, one or more of the loops may protrude therefrom such that the loops are readily able to receive a user's fingers (e.g. for use in unfolding of the disposal device from its folded-up state).

The container may have a height of between 5 cm and 25 cm and a width of between 2 cm and 12 cm. The container may itself be perfumed or contain a perfume as desired.

The container may be made of an impervious material that is resistant to the transmission of soil and odors from the soiled item. For example, the container may be made of a plastic or waxed material. When used for the disposal of sanitary products, the container can be impervious to aqueous liquid materials.

Although the container may be rigid, it can be deformable so as to be readily portable. Accordingly, the container may be folded or rolled into a smaller volume for ease of transport and storage.

In one embodiment, the container used in the disposal of sanitary products is opaque. However, for other applications, for example, in the disposal of medical products or food products, the container may be translucent or transparent as desired. The container may be colored or colorless. The container may have printed subject matter applied thereto (e.g. to one or more walls thereof).

In some embodiments, the disposal device herein is also configured to act as a packaging device for the item in its pre-soiled state. Thus, in one usage scenario the item is supplied within the packaging and disposal device in a pre-usage state; then removed from the packaging and disposal device and used in some way, which usage results in soiling thereof; and then the soiled item is placed into the packaging and disposal device for disposal thereof.

In some embodiments, the packaging and disposal device is provided with a second seal, which typically is located to seal off access to the mouth. Thus, to access an item within the packaging and disposal device the second seal must be broken to allow access to and opening of the mouth such that the item may be removed from the device through the opened mouth.

In some embodiments, the second seal is configured to be non-resealable (i.e. once the second seal is broken it may not be resealed). In some embodiments, the second seal is a tear-off (i.e. tear-away) seal, which typically has perforations to guide the tearing-off process. Suitably, the second seal is a tamper-evident seal. In other embodiments, the second seal is a re-sealable seal.

The present invention also extends to a method of making the disposal device described above. The present invention further extends to a method of using the disposal device described above.

The present invention further extends to a dispenser device for dispensing the disposal device herein.

The dispenser device may include a housing which is arranged to accommodate plural disposal devices herein, each of which is in a “storage” configuration. The dispenser device may enable dispensing of one of said plural disposal devices at a time (i.e. serial dispensing). The dispenser device may be arranged such that on dispensing the mouth manipulation means (e.g. one or more loops) is presented to the user for ready interaction with the user's fingers and/or thumb. The dispenser device may be shaped for ready accommodation in the user's hand (e.g. within the user's palm) and for such ready interaction of the user's fingers and/or thumb with a mouth manipulation means of the disposal device that is being dispensed therefrom.

In one aspect, the dispenser device is arranged for stacked storage (i.e. one of top of the other) of plural disposal devices herein. In the stacking arrangement each disposal device may lie flat, although the uppermost disposal device of the stack (i.e. that one which has no other disposal device lying thereon) may be arranged such as to present at least one manipulation means away therefrom for ready interaction with the user's fingers and/or thumb. The dispenser device may further include one or more flaps or other suitable means for compressing the disposal devices in the stack to ensure maintenance of the flat stacking arrangement and/or to ensure that only one disposal device is dispensed at a time.

In another aspect, the dispenser device may be arranged for rolled-up storage (i.e. in a rolled configuration) of plural disposal devices herein. Suitably, the plural disposal devices are arranged in a joined up series (i.e. the tail end of one joining to the lead end of another at a joint) with said joint being broken as one disposal device is torn or ripped off from the roll on dispensing thereof. The lead disposal device of the roll (i.e. that one which is outermost in the roll) may be arranged such as to present at least one manipulation means away therefrom for ready interaction with the user's fingers and/or thumb. A “snail shell” form dispenser is herein envisaged.

### BRIEF DESCRIPTION OF THE DRAWINGS

FIG. 1 is a perspective view of a first embodiment of a disposal device of the present invention in the closed position;

FIG. 2 is a perspective view of the disposal device of FIG. 1 in the open position;

FIG. 3 is a perspective view of a second embodiment of a disposal device of the present invention in the closed position;

FIG. 4 is a perspective view of the disposal device of FIG. 3 in the open position;

FIG. 5 is an enlarged view of a section of an embodiment of a seal to the mouth of the container of the present invention;

FIG. 6 is a further view of the type of seal shown in FIG. 5; and

FIG. 7 is an enlarged view of a section of an alternative seal to the mouth of the container of the present invention;

FIG. 8 is a perspective view of a third embodiment of a disposal device of the present invention in the lips and mouth closed position;

FIG. 9 is a side view of the third embodiment of FIG. 8 in the lips open, but mouth closed position;

FIG. 10 is a perspective view of the top part of the third embodiment of FIG. 8 shown in combination with a user's thumb/finger in the lips open, but mouth closed position of FIG. 9;

FIG. 11 is a view from above of the top part of the third embodiment of FIG. 8 shown in combination with a user's thumb/finger in the lips open, but mouth closed position of FIG. 9;

FIG. 12 is a view from above of the top part of the third embodiment of FIG. 8 shown in combination with a user's thumb/finger in a lips open and mouth part-open position;

FIG. 13 is a view from above of the top part of the third embodiment of FIG. 8 shown in combination with a user's thumb/finger in a lips open and mouth wide-open position;

FIG. 14 is a perspective view of a fourth embodiment of a disposal device of the present invention in the lips and mouth closed position;

FIG. 15 is a perspective view of a fifth embodiment of a disposal device of the present invention in the lips and mouth closed position;

FIG. 16 is a perspective view of a sixth embodiment of a disposal device of the present invention in the mouth closed position;

FIG. 17 is a side view of the sixth embodiment of FIG. 16;

FIGS. 18a and 18b show perspective views of a seventh embodiment of a disposal device of the present invention in respectively mouth closed and mouth open positions;

FIG. 19 shows a perspective view of an eighth embodiment of a disposal device of the present invention in the mouth closed position;

FIG. 20 shows a perspective view of a first dispenser for dispensing a disposal device of the present invention;

FIGS. 21a to 21c show perspective views of a second dispenser for dispensing a disposal device of the present invention in respectively “dispenser closed”, “dispenser part-open” and “dispenser open” positions;

FIGS. 22a to 22d show an eighth disposal device herein in successive stages during a typical use operation;

FIGS. 23a to 23f show side views of a ninth disposal device herein during successive stages of a folding-up operation thereof;

FIGS. 24a and 24b show side views of first and second packaging and disposal devices herein;

FIGS. 25a to 25c show side views of third to fifth packaging and disposal devices herein;

FIG. 26 shows a side view of a sixth packaging and disposal device herein; and

FIGS. 27a and 27b show side views of seventh and eighth packaging and disposal devices herein.

### DETAILED DESCRIPTION OF EXEMPLARY EMBODIMENTS OF THE INVENTION

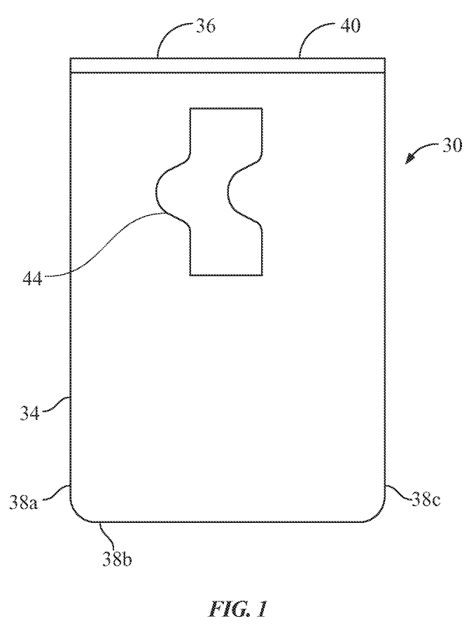

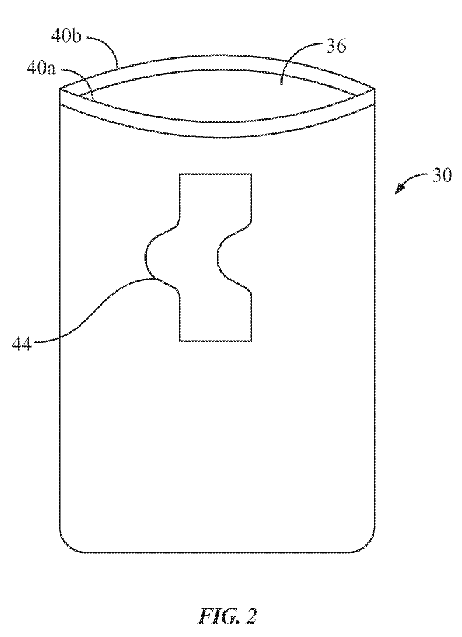

FIGS. 1 and 2 show perspective views of an embodiment of a disposal device 30. The disposal device is made up of a container 34 having a mouth 36. In the embodiment shown in FIGS. 1 and 2, the mouth 36 forms part of the container. However, it is possible that the mouth 36 may form a separate section that is attached to the container 34 to form the disposal device 30.

In the embodiment shown in FIGS. 1 and 2, the container is formed in the shape of a rectangle, three edges 38a, 38b, 38c of which are sealed. The mouth is formed along the fourth edge 40 of the container 34.

Mouth manipulation means 42 are provided on one or both sides of the mouth 36. It will be appreciated that there are many variations of mouth manipulation means that may be used. For example, the container may be provided with a loop on one side of the mouth on the outside of the container through which one or more fingers, or a thumb, could be inserted and a strip of adhesive on the outside of the container on the other side of the mouth. Thus, in use, a person could adhere their finger(s) or thumb to one side of the container, and use the loop to pull apart the other side, therefore moving the container into an open position. Once a soiled item has been placed inside the container, the two parts of the mouth may be pushed together into a closed position.

In the embodiment shown in FIGS. 1 and 2, the container is provided with two loops, a first loop 44 on a first exterior side of the container and a second loop (not shown) on an opposing side, such that the loops are located on the outside of the container on each side of the mouth 36 respectively. The loops 44 are arranged such that one or more fingers or a thumb may be inserted through each loop to enable a person to be able to open or close the container by pulling apart their fingers or thumb to open the container and pushing them together to close the container. Typically, the user's thumb is received by a first loop 44 and user's index finger by a second loop (not shown) on the opposing side of the disposal device 30 and the opening/closing action is by spacing/de-spacing action of the thumb and index finger.

The container may be made of any material that is substantially impervious and is resistant to the transmission of soil and odors from the soiled item. For example, the container may be made of a plastic polymer or waxed material. When used for the disposal of sanitary products, the container should generally be impervious to aqueous liquid materials.

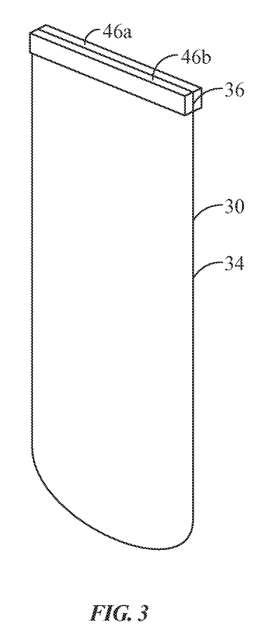

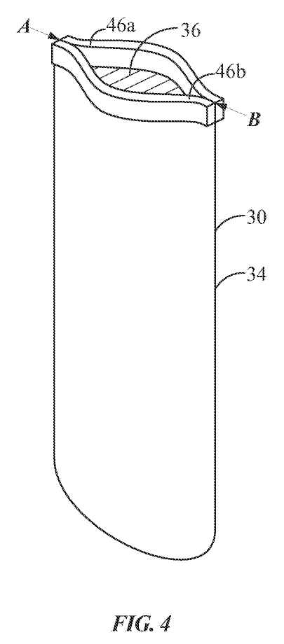

FIGS. 3 and 4 show an alternative embodiment of the disposal device 30 where the mouth 36 is closed and defined by rigid deformable strips 46a, 46b. The strips 46a, 46b are attached to, or form an integral part of the container. The strips 46a, 46b define a mouth 36 through which soiled items may be received by the container.

The disposal device, as shown in FIG. 3, is in an unbiased, closed position where the strips 46a, 46b abut each other to form a seal. In FIG. 4, the strips 46a, 46b are biased apart to an open position of the mouth 36 through which soiled items may be deposited in the container.

The strips 46a, 46b are biased apart by the manual application of force on the ends of the strips 46a, 46b in the direction of arrows A and B so that the strips flex into facing arcs to form the aperture. Upon removal of the force applied the strips 46a, 46b return to the closed position.

In each of FIGS. 1 to 4, a hygienic seal may be provided by lining the internal boundary of the mouth 36 with adhesive (not shown). The adhesive may be placed on just one internal boundary, or both. Thus, in FIGS. 1 and 2, after a soiled item (not shown) has been placed in the container 34, when the two boundaries of the mouth 36 of the container 34 are pressed together, they will adhere to one another to form a hygienic seal (not shown). Similarly, in the container 34 shown in FIGS. 3 and 4, upon release of manual pressure biasing the strips 46a, 46b apart to form an aperture, the strips 46a, 46b return to the closed position and become adhered to one another by the adhesive.

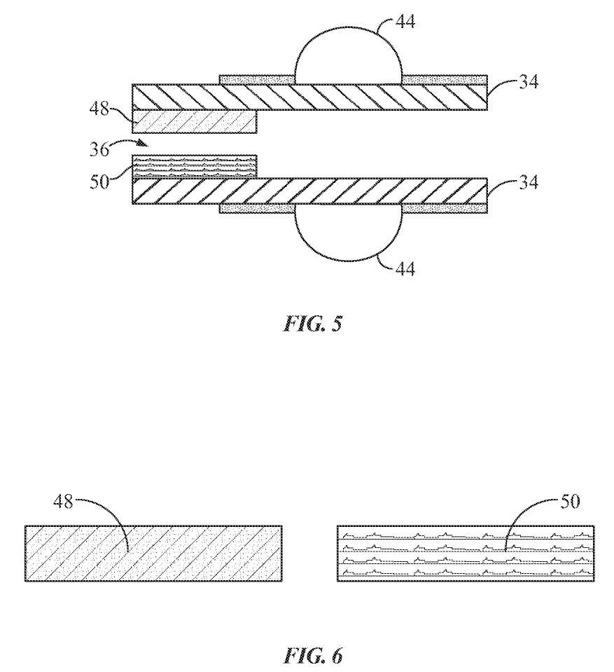

FIGS. 5, 6 and 7 show two further variations on the type of seal that may be used. FIGS. 5 and 6 illustrate a section through an embodiment of the mouth 34 of the container 34 having a strip of material 48 provided on one inside of the boundary of the mouth 36. This is also illustrated in FIG. 6 where a different, but complementary strip of material 50 that is capable of bonding to the first material is provided on the inside boundary of the other side of the mouth 36. These different materials, when brought together, form a seal (not shown). The two different materials are arranged to form a hook and loop arrangement to engage together

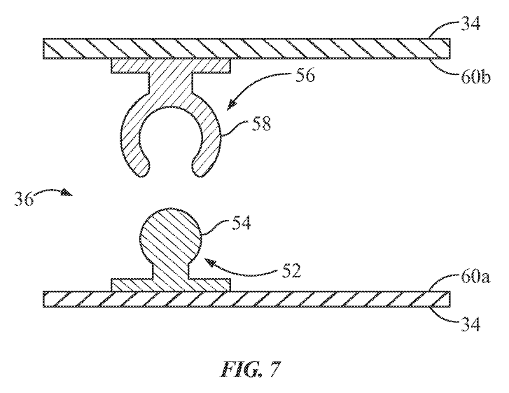

FIG. 7 shows a section through a tongue and groove arrangement. A first strip 52 with a tongue 54 provided along its length is located on the inside boundary of a first side 60a of the container 34 defining the mouth 36. A second strip 56 provided with a corresponding groove 58 along its length locates on the other opposing second side 60b of the container 34 defining the mouth 36. Thus, when the two sides 60a, 60b about the mouth 36 are pressed together in the closed position, the tongue 54 and groove 58 respectively engage together to form a seal (not shown).

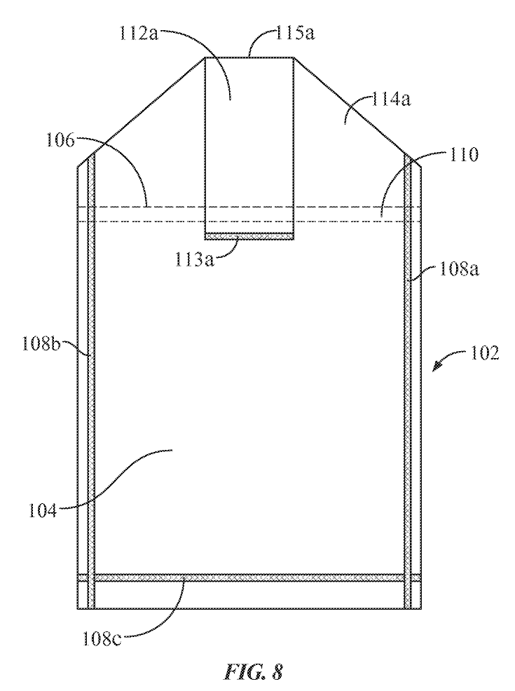

FIGS. 8 and 9 show a third embodiment of a disposal device 102 herein, which adopts the storage form of a flat pouch, and in an in-use configuration is suitable for the disposal of a soiled item (not shown). The disposal device defines a container 104, which is formed by the joining together of two flat plastic polymer sheets 103a, 103b by means of first, second and third permanent seal lines 108a, 108b and 108c and adhesive seal line 110. The adhesive seal line 110 defines a reversibly sealable (i.e. unseal/seal) barrier to the container 104 and the top (i.e. outer) edge thereof generally corresponds to the mouth 106 of the container 104.

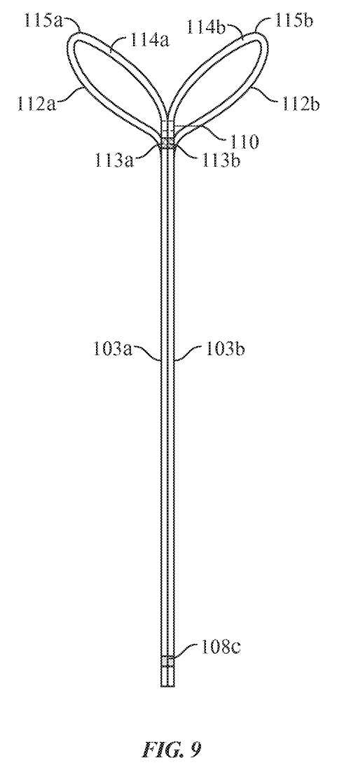

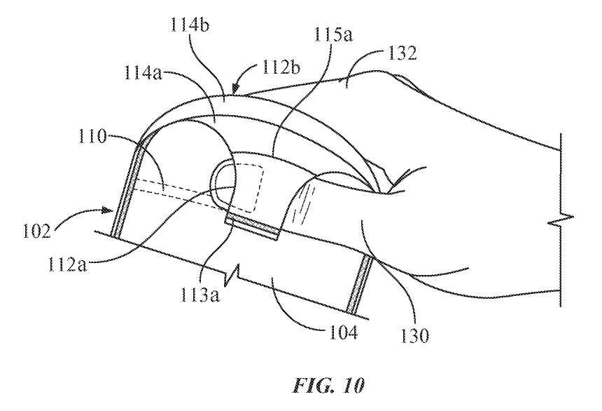

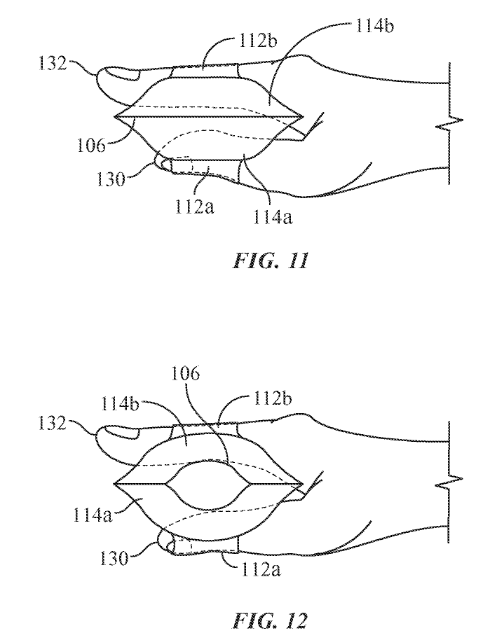

First and second lips 114a, 114b are provided to the mouth 106 and these are moveable to a “lips open” configuration (see FIGS. 10 and 11) in which the lips form a guide for the receipt of a soiled item through the mouth 106 and into the container 104. First and second loops 112a, 112b are further provided to the mouth 106 and lips 114a, 114b. It will be noted that each loop 112a, 112b extends from an outer edge 115a, 115b of a respective lip 114a, 114b and attaches to an outside wall of the container 104 by means of respective permanent seal line 113a, 113b. It will further be noted that each permanent seal line 113a, 113b runs parallel to the adhesive seal line 110 and locates just underneath that adhesive seal line 110. It will be further noted that the loop profile (i.e. the shape of the loop aperture) defined by each loop 112a, 112b is generally elongate (i.e. elongate oval) to enable ready receipt of a user's thumb/fingers.

FIGS. 10 and 11 show the disposal device of FIGS. 8 and 9 at a first stage of a use operation. First loop 112a receives the user's thumb 130 and second loop 112b receives the user's index finger 132. The lips 114a, 114b are thereby, partly opened to give access to the mouth 106, which remains closed at this first stage.

FIG. 12 shows a second stage in the use operation, in which the user's thumb 130 and index finger 132 have been separated somewhat to cause the seal 110 to be broken and to allow the mouth 106 to open slightly. The lips 114a, 114b are now wide open and provide a guiding passage for the insertion of a soiled item into the mouth 106.

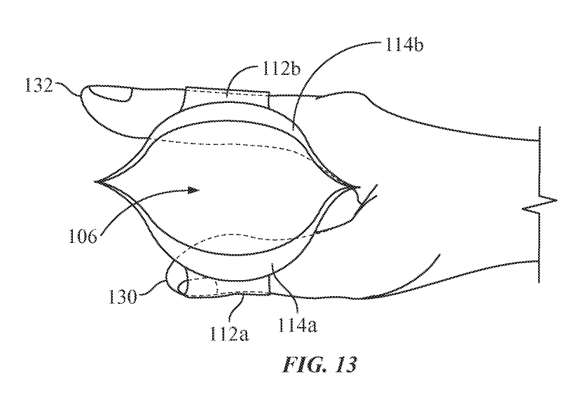

FIG. 13 shows a third stage in the use operation, in which the user's thumb 130 and index finger 132 have been separated more widely to cause the mouth 106 to open broadly. The lips 114a, 114b are now even more wide open and continue to provide a guiding passage for the insertion of a soiled item into the now gaping mouth 106.

Once the soiled item (not shown) has been placed into the container 104 through the mouth 106 thereof the user will bring his/her thumb 130 and index finger 132 back together again. That is to say, the top of disposal device will return via a “mouth slightly open” position as shown at FIG. 12 to a “mouth closed” position as shown at FIGS. 10 and 11. The user then employs his/her thumb 130 and index finger 132 to press against the adhesive seal line 110 to reseal the container 104. The soiled item is thereby, contained in sealed fashion within the container 104.

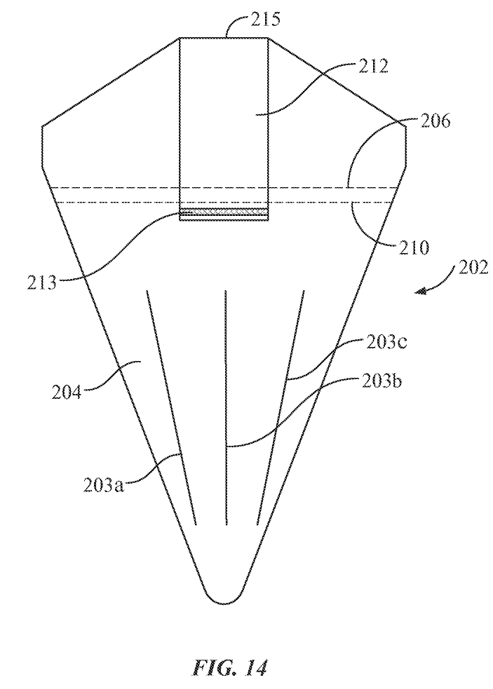

FIG. 14 shows a fourth embodiment of a disposal device 202 herein, which may be appreciated to be a variation of the third embodiment of FIGS. 8 to 13. The disposal device 202 defines a container 204, which is formed by a tapering bag defined in part by tapered fold lines 203a, 203b, 203c. As before, adhesive seal line 210 defines a reversibly sealable (i.e. unseal/seal) barrier to the container 204 and the top (i.e. outer) edge thereof generally corresponds to the mouth 206 of the container 204.

Lips 214 are again provided to the mouth 206 and as before, these are moveable to a “lips open” configuration in which the lips form a guide for the receipt of a soiled item through the mouth 206 and into the container 204. Loops 212 are further provided to the mouth 206 and lips 214. As before, it will be noted that each loop 212 extends from an outer edge 215 of a respective lip 214 and attaches to an outside wall of the container 104 by means of respective permanent seal line 213. It will further be noted that each permanent seal line 213 runs parallel to the adhesive seal line 210 and locates just underneath that adhesive seal line 210.

The general operation of the fourth embodiment of FIG. 14 closely mirrors that of the third embodiment of FIGS. 8 to 13 and is therefore, not described further.

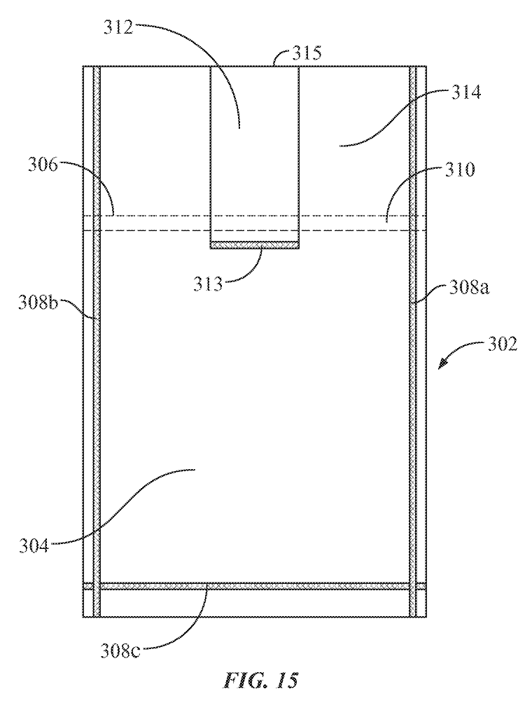

FIG. 15 shows a fourth embodiment of a disposal device 302 herein, which may be appreciated to be another variation of the third embodiment of FIGS. 8 to 13. The disposal device 302 adopts the storage form of a flat pouch and in an in-use configuration is suitable for the disposal of a soiled item (not shown). The disposal device defines a container 304, which is formed by the joining together of two flat plastic polymer sheets by means of first, second and third permanent seal lines 308a, 308b and 308c and adhesive seal line 310. The adhesive seal line 310 defines a reversibly sealable (i.e. unseal/seal) barrier to the container 304 and the top (i.e. outer) edge thereof generally corresponds to the mouth 306 of the container 304.

Lips 314 are provided to the mouth 306 and these are moveable to a “lips open” configuration in which the lips form a guide for the receipt of a soiled item through the mouth 306 and into the container 304. Loops 312 are further provided to the mouth 306 and lips 314. It will be noted that each loop 312 extends from an outer edge 315 of a respective lip 314 and attaches to an outside wall of the container 304 by means of respective permanent seal line 313. It will further be noted that each permanent seal line 313 runs parallel to the adhesive seal line 310 and locates just underneath that adhesive seal line 310. The loop profile (i.e. the shape of the loop aperture) defined by each loop 312 is generally elongate (i.e. elongate oval) to enable ready receipt of a user's thumb/fingers.

The general operation of the fifth embodiment of FIG. 15 again closely mirrors that of the third embodiment of FIGS. 8 to 13 and is therefore, not described further.

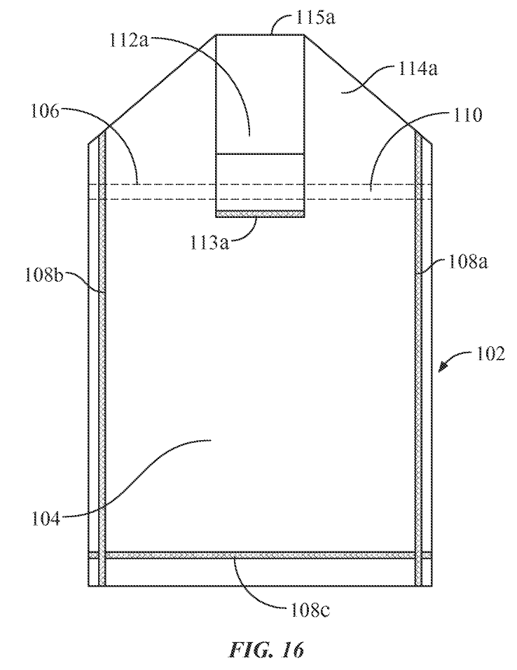

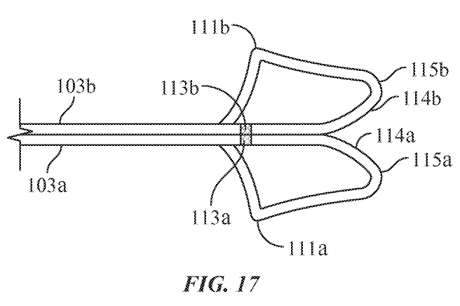

FIG. 16 shows a fifth embodiment of a disposal device 102 herein, which is identical to the third embodiment of FIGS. 8 to 13 other than that an additional fold 111a, 111b is provided to each loop 112a, 112b. As may be seen by reference to FIG. 17, which shows a top detail of the disposal device, the consequence of having this additional fold 111a, 111b is that each loop 112a, 112b tends to “pop up” into a lips 114a, 114b semi-open position in the absence of any pressure on the loop 112a, 112b. Thus, in effect the additional fold 111a, 111b tends to bias each loop 112a, 122b into such a semi-open position.

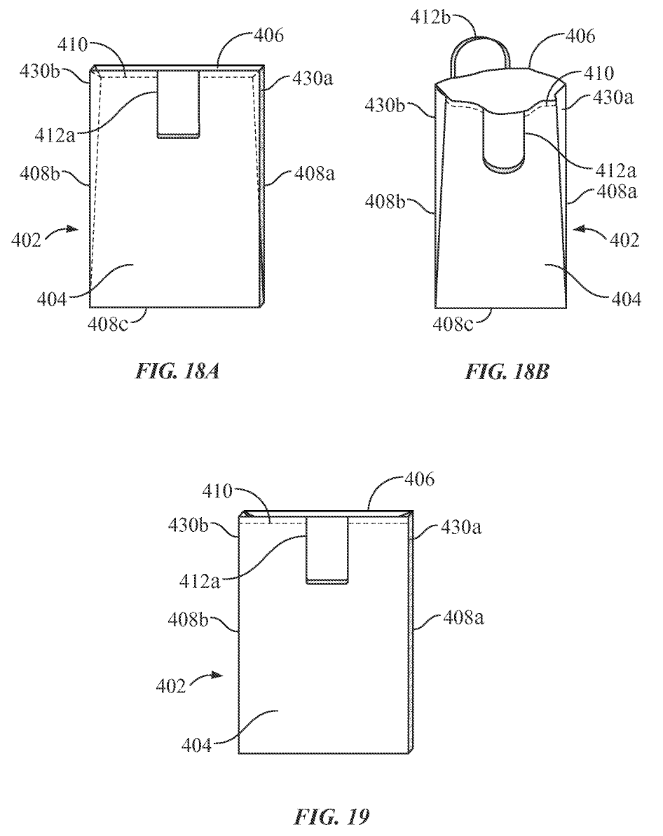

FIGS. 18a and 18b show a sixth embodiment of a disposal device 402 herein, which defines a rectangular pouch-form container 404. As seen at FIG. 18a, the disposal device 402 adopts the storage form of a flat pouch. As seen at FIG. 18b, an in-use configuration is suitable for the disposal of a soiled item (not shown). The rectangular pouch-form container 404 has major edges 408a, 408b which define its length; a lower minor edge 408c, which defines its base; and adhesive seal line 410. The adhesive seal line 410 defines a reversibly sealable (i.e. unseal/seal) barrier to the container 404 and the top (i.e. outer) edge thereof generally corresponds to the mouth 406 of the container 404. Loops 412a, 412b are provided to the mouth 406. The loop profile (i.e. the shape of the loop aperture) defined by each loop 412 is generally elongate (i.e. elongate oval) to enable ready receipt of a user's thumb/fingers.

Gussets 430a, 430b are provided at each major edge 408a, 408b of the container 404. The use of such gussets 430a, 430b is advantageous in that the effective volume of the container 404 for soiled item disposal is increased. It will be noted that each gusset 430a, 430b is inclined at angle of about 10° to its respective edge 408a, 408b such that the gusset 430a, 430b tapers towards the bottom of the pouch 404. It will be noted that the top part of each gusset 430a, 430b lies just below the adhesive seal line 410.

FIG. 19 shows a seventh embodiment herein, which is identical to the sixth embodiment of FIGS. 18a and 18b other than that each gusset 430a, 430b runs parallel to its major edge 408a, 408b (i.e. it is not tapered).

The general principle of operation of the sixth and seventh embodiments of respectively FIGS. 18a and 18b and FIG. 19 closely mirrors that of the previously described embodiments and is therefore, not described further.

Any of the first to seventh embodiments described above is suitable for the disposed receipt of an item such as a condom, tampon, sanitary towel, medical item (not shown) for disposal thereof, or for the disposed receipt of a medical or forensic item (not shown) for later analysis thereof.

FIG. 20 shows a first dispenser device 550 herein that is suitable for storage of plural disposal devices in a stacking arrangement and which enables serial (i.e. one at a time) dispensing of disposal devices from the stack.

The first dispenser device 550 comprises a rectangular box housing 552 (e.g. formed of cardboard). The housing 552 is provided with an opening 554 (which may in aspects, initially be provided with a tear-off cover such as a perforated seal) through which a loop 512 of the lead disposal device 502 of the stack projects. In use, the user would hold the box housing 552 in a cupped hand in such a way as to allow for ready insertion of a finger or thumb into the projecting loop 512 of the lead disposal device 502. Once such finger/thumb has been inserted into the loop 512 the user would then pull the loop 512 upwards to thereby dispense the disposal device 502 from the box housing 552. The box housing 552 is further provided with an internal flap 556, which is arranged to apply downward pressure to the stack and thereby to both keep the disposal devices 502 in the stack in a flat configuration and to ensure that only one disposal device 502 is dispensed at a time.

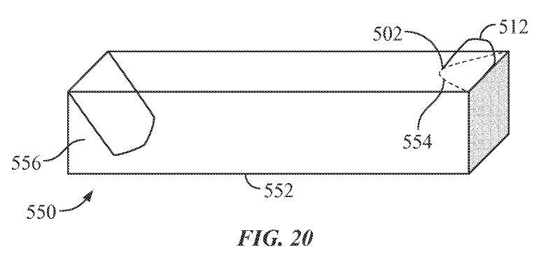

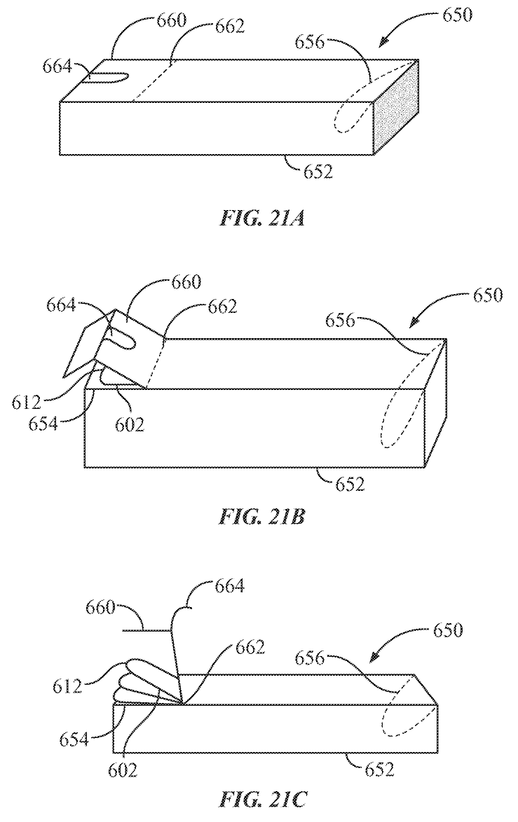

FIGS. 21a to 21c shows a second dispenser device 650 herein in successive states of operation. The second dispenser device 650 is suitable for storage plural disposal devices 602 in a stacking arrangement and which enables serial (i.e. one at a time) dispensing of disposal devices from the stack.

The second dispenser device 650 comprises a rectangular box housing 652 (e.g. formed of cardboard). The housing 652 is provided with an opening 654 that is provided with a lid 660. The lid 660 joins to the housing at fold 662 about which it hinges upwards into its lid open state (as shown at FIGS. 21b and 21c) tear-off. The lid 660 is further provided with an upwardly projecting tab 664, which assists in pulling open of the lid 660. Optionally and not shown, the housing 652 may be provided with an extra piece of cardboard into which the lid 660 folds in a “lid closed” position. When the lid 660 is in its “lid open” position loop 612 of the lead disposal device 602 of the stack projects from the opening 654. In use, the user would hold the box housing 652 in a cupped hand in such a way as to allow for pulling up of the tab 664 to open the lid 660. A finger or thumb would then be inserted into the projecting loop 612 of the lead disposal device 602. Once such finger/thumb has been inserted into the loop 612 the user would then pull the loop 612 up and away from the box housing 652 to thereby dispense the lead disposal device 602. The box housing 652 is further provided with an internal flap 656, which is arranged to apply downward pressure to the stack and thereby to both keep the disposal devices 602 in the stack in a flat configuration and to ensure that only one disposal device 602 is dispensed at a time.

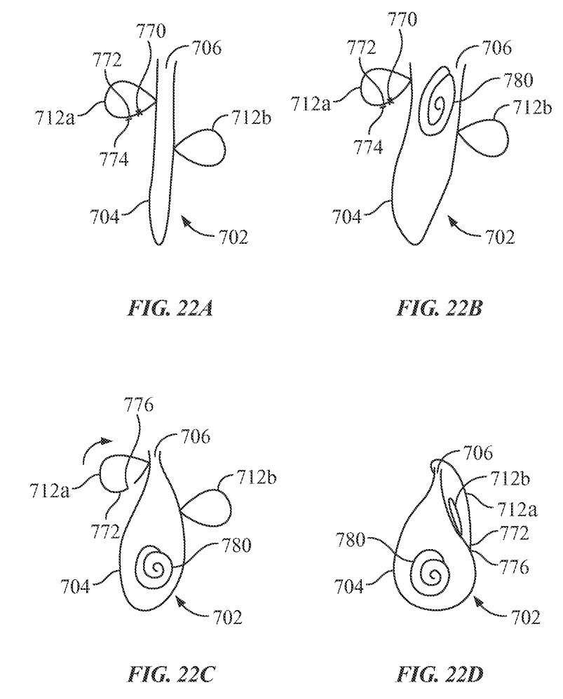

FIGS. 22a to 22d show an eighth disposal device 702 herein during successive stages of a typical use operation.

Referring initially to FIG. 22a it will be seen that the eighth disposal device 702 comprises a bag 704 to which first 712a and second 712b loops are provided. It will be noted that the first loop 712a locates closer to the mouth 706 of the bag than does the second loop 712b. The first loop 712a is further provided with a perforated portion 770 and an adhesive portion 772 protected by a pull over cover strip 774.

Referring now to FIG. 22b, the mouth 706 of the bag 704 has been opened by pulling apart the loops 712a, 712b to enable receipt of a rolled-up, soiled sanitary napkin 780.

At FIG. 22c, the sanitary napkin 780 is fully-received within the bag 704 and the mouth 706 closed. The perforated seal 772 is now broken to sever the first loop 712a, and the cover strip 774 is removed from the adhesive portion 722. The loose end 776 of the broken first loop 712a is now brought up and over the mouth 706 to the position as shown in FIG. 22d where the broken first loop 712a lies over the flattened second loop 712b and seals to a wall of the bag 704 by means of adhesive portion 772. Thus, the soiled sanitary napkin is enclosed by and sealed within the bag 704.

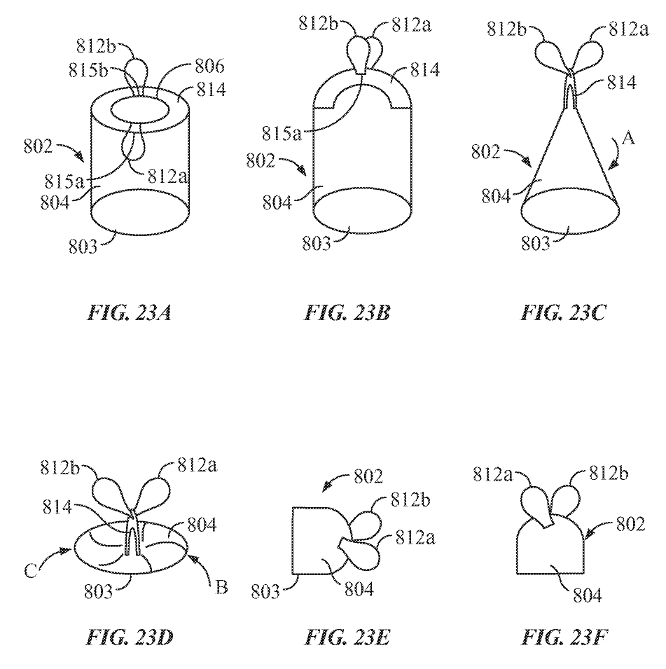

FIGS. 23a to 23f illustrate a ninth disposal device herein, which is arranged to be supplied in a folded-up configuration for unfolding thereof by a user into an unfolded usage configuration. FIGS. 23a to 23f illustrate successive stages of the unfolding operation in reverse (i.e. FIG. 23a is the unfolded device in the usage configuration and FIG. 23f is the device as it would be supplied in its folded-up state).

In more detail, FIG. 23f shows the disposal device 802 with its bag container 804 in a folded-up state, and with first and second finger loops 812a, 812b protruding upwards therefrom. It will be appreciated that such folded-up state of the disposal device might be readily accommodated in a dispenser device such as one based on a variation of the dispenser devices of FIGS. 20 to 21c. In FIG. 23e, the disposal device is shown again in its container 804 folded-up state, but now with the finger loops 812a, 812b protruding sideways therefrom.

To understand how the disposal device is transformed into the folded-up state of FIGS. 23e and 23f, it is most convenient to start with the disposal device 802 in its usage configuration of FIG. 23a. It will be appreciated that the unfolding operation will simply be the reverse of the folding-up operation as now described in more detail.

FIG. 23a shows that the container 804 defines a circular base 803, and is provided with a mouth 806 defined by a circular lip 814. In other embodiments, the lip 814 is ovular. The first and second finger loops 812a, 812b are provided to opposing edges 815a, 815b of the lip 814. In use, the first and second finger loops 812a, 812b are used to manipulate the lip to a “lip open” configuration as shown in FIG. 23a, in which the lip 814 forms a guide for the receipt of a soiled item through the mouth 806 and into the container 804. The lip 814 is further provided with sealing material (e.g. sticky) on the inner surface thereof such that once a soiled item has been received within the container 804 the mouth 806 may be sealed by bringing the opposing edges 815a, 815b of the inner surface of the lip 814 together (e.g. by a finger-thumb action of the first and second finger loops 812a, 812b).

FIG. 23b shows the disposal device 802 in a partly folded-up state, in which the opposing edges 815a, 815b of the lip 814 have been brought together such that the lip 814 folds onto itself and the finger loops 812a, 812b lie side-by-side.

FIG. 23c shows the disposal device 802 in a subsequent partly folded-up state, in which the finger loops 812a, 812b and lip 814 are twisted in a rotational sense (see Arrow A) about an axis extending from the centre of the circular base 803 such that the container 804 begins to adopt a twisted-up configuration. In other embodiments, the base is ovular.

FIG. 23d shows the disposal device 802 in a further subsequent partly folded-up state, in which the twisted-up container 804 has been pushed down towards the base 803, and in which the finger loops 812a, 812b and lip 814 stand proud from the (now flattened) container 804 and base 803.

To now transform the disposal device from its state of FIG. 23d to the fully folded-up state of FIG. 23e, the circular base 803 is folded about itself (as shown by Arrows B and C) such as to create a half-moon, within which locates the twisted-up container 804, and from which only the finger loops 812a, 812b protrude.

As previously mentioned, unfolding of the disposal device from its folded-up state of FIGS. 23e and 23f to its usage configuration of FIG. 23a simply requires following the folding operation in reverse. The user's fingers (as accommodated within the finger loops 812a, 812b) will in use, guide this unfolding operation.

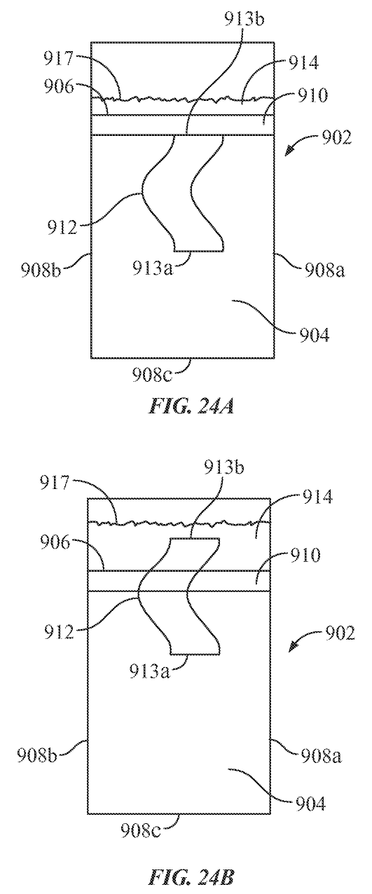

FIGS. 24a and 24b show first and second packaging and disposal devices 902, which may be appreciated to be close variations of one another. Both devices are suitable for the hygienic packaging and subsequent disposal of an item such as a condom, tampon or sanitary towel (not shown) or for the hygienic packaging and storage for layer analysis of a medical or forensic swab.

In more detail, the packaging and disposal devices 902 of FIGS. 24a and 24b adopt the storage form of a flat pouch, and in an in-use configuration that is suitable for the disposal of a soiled item (not shown). Each packaging and disposal device defines a container 904, which is formed by the joining together of two flat plastic polymer sheets by means of edge seals 908a, 908b and 908c and adhesive seal line 910. The adhesive seal line 910 defines a reversibly sealable (i.e. unseal/seal) barrier to the container 904 and the top (i.e. outer) edge thereof generally corresponds to the mouth 906 of the container 904.

Opposing lips (only one side visible) 914 are provided to the mouth 906 and these are moveable to a “lips open” configuration, in which the lips form a guide for the receipt of a soiled item through the mouth 906 and into the container 904. First and second loops (only one visible) 912 are further provided to the mouth 906 and lips 914. It will be noted that the positions of the loops 912 is slightly different for the first packaging and disposal device of FIG. 24a compared to that of the second packaging and disposal device of FIG. 24b. As shown in FIG. 24a, each loop 912 attaches between two points 913a, 913b of an outside wall of the container 904 and below the adhesive seal line 910. As shown in FIG. 24a, each loop 912 attaches between two points 913a, 913b of an outside wall of the container 904, one of which is above and the other below the adhesive seal line 910. It will be noted that the loop profile (i.e. the shape of the loop aperture) defined by the loops 912 of each packaging and disposal device is arranged to enable ready receipt of a user's thumb/fingers.

Both of the first and second packaging and disposal devices also comprise a tear-off seal 917, located external to the mouth 906, which acts to hygienically seal off the interior of the container 904 until the tear-off seal 917 is broken. The tear-off seal 917 may also be appreciated to be tamper-evident.

In a typical usage scenario, the first and second packaging and disposal devices are supplied with an item (e.g. a condom, tampon or sanitary towel, not shown) locating within the container 904. To access that item for use thereof, the tear-off seal 917 is broken such that the item may then be accessed by opening the mouth 906 (e.g. using the loops 912 as mouth-opening means). The item is then used, and such usage results in soiling thereof. The soiled item is then replaced into the container 904, by opening the lips 914 thereof by means of the loops (e.g. by a finger-thumb action) and placing the item through the mouth 906. Once the soiled item has been received into the container 904, the lips 914 are closed and the container 904 sealed off by means of the adhesive seal 910. The soiled item may then be hygienically disposed of.

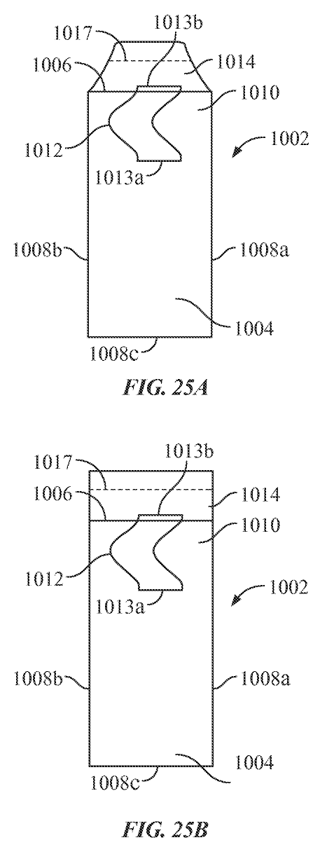

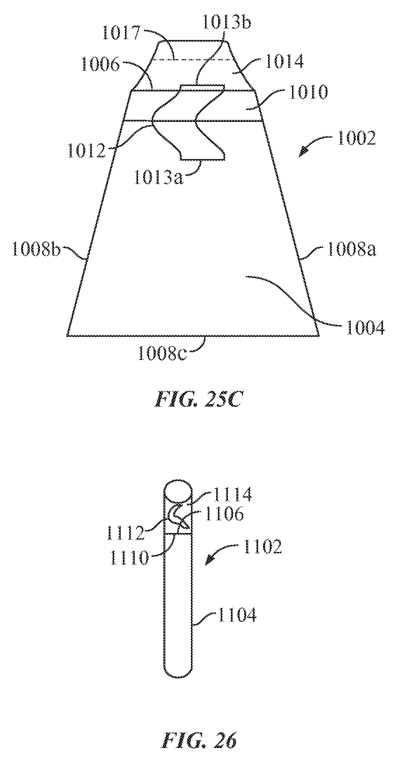

FIGS. 25a to 25c show third, fourth and fifth packaging and disposal devices 1002, any of which may be appreciated to be a close variation of the other. Any of these devices is suitable for the hygienic packaging and subsequent disposal of an item such as a condom, tampon, tampon applicator or sanitary towel (not shown) or for the hygienic packaging and storage for layer analysis of a medical or forensic swab.

In more detail, the packaging and disposal devices 1002 of FIGS. 25a to 25c adopt the storage form of a flat pouch, and in an in-use configuration that is suitable for the disposal of a soiled item (not shown). Each packaging and disposal device defines a container 1004, which is formed by the joining together of two flat plastic polymer sheets by means of edge seals 1008a, 1008b and 1008c and adhesive seal line 1010. The container 1004 of the third and fourth packaging and disposal devices of FIGS. 25a and 25b is rectangular, whereas that of the fifth packaging and disposal device of FIG. 25c is tapered in form. The container 1004 may be provided with gusseting. The adhesive seal line 1010 defines a reversibly sealable (i.e. unseal/seal) barrier to the container 1004 and the top (i.e. outer) edge thereof generally corresponds to the mouth 1006 of the container 1004.

Opposing lips (only one side visible) 1014 are provided to the mouth 1006 and these are moveable (once released, see later) to a “lips open” configuration, in which the lips form a guide for the receipt of a soiled item through the mouth 1006 and into the container 1004. First and second loops (only one visible) 1012 are further provided to the mouth 1006 and lips 1014. Each loop 1012 attaches between two points 1013a, 1013b of an outside wall of the container 1004, one of which is above and the other below the adhesive seal line 1010. It will be noted that the loop profile (i.e. the shape of the loop aperture) defined by the loops 1012 of each packaging and disposal device is arranged to enable ready receipt of a user's thumb/fingers.

The third to fifth packaging and disposal devices also comprise a tear-off seal 1017, which locates outer to the mouth 1006 and acts to hygienically seal off the interior of the container 1004 until the tear-off seal is broken. This tearing off action acts to release the lips 1014, and also the loops 1012, which otherwise lie essentially flat against the container 1004. It will be noted that the shape of the uppermost part, and in particular of the lips 1014, is slightly different for the third and fifth packaging and disposal devices 1002 of FIGS. 25a and 25c compared to that of the fourth packaging and disposal device 1002 of FIG. 25b. The lips 1014 of the third and fifth packaging and disposal devices 1002 are tapered in form, whereas those lips 1014 of the fourth packaging and disposal device are square cut.

In a typical usage scenario, the third, fourth or fifth packaging and disposal devices are supplied with an item (e.g. a condom, tampon or sanitary towel, not shown) locating within the container 1004. To access that item for use thereof, the tear-off seal 1017 is broken such that the item may then be accessed by opening the mouth 1006 (e.g. using the loops 1012 as mouth-opening means). The item is then used, and such usage results in soiling thereof. The soiled item is then replaced into the container 1004, by opening the lips 1014 thereof by means of the loops (e.g. by a finger-thumb action) and placing the item through the mouth 1006. Once the soiled item has been received into the container 1004, the lips 1014 are closed and the container 1004 sealed off by means of the adhesive seal 1010. The soiled item may then be hygienically disposed of.

FIG. 26 shows a sixth packaging and disposal device 1102 herein that may be appreciated to be a variation of those already described. The container 1104 is formed of a hard plastic polymer (e.g. from recycled stock). The upper part of the device (i.e. that part above the adhesive seal 1110) including the mouth 1106, lips 1014 and loops 1112 are formed of a soft and malleable plastic polymer material.

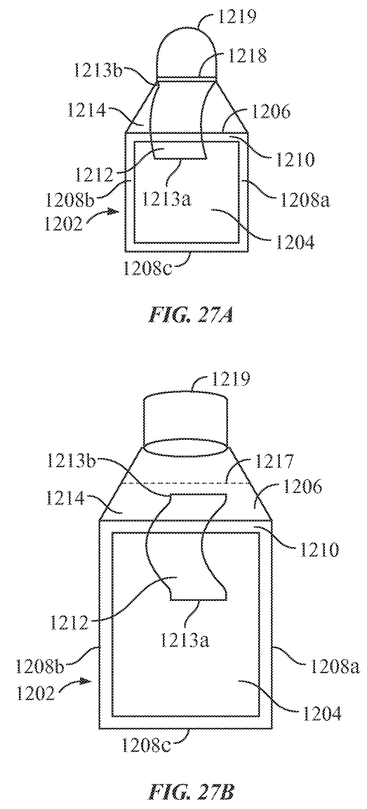

FIGS. 27a and 27b show seventh and eighth packaging and disposal devices 1202, which may be appreciated to be close variations of each other. Either of these devices is suitable for the hygienic packaging and subsequent disposal of an item such as a condom, tampon, tampon applicator or sanitary towel (not shown).

In more detail, the packaging and disposal devices 1202 of FIGS. 27a and 27b adopt the storage form of a flat pouch, and in an in-use configuration that is suitable for the disposal of a soiled item (not shown). Each packaging and disposal device defines a rectangular container 1204, which is formed by the joining together of two flat plastic polymer sheets by means of edge seals 1208a, 1208b and 1208c and adhesive seal line 1210. The container 1204 may be provided with gusseting. The adhesive seal line 1210 defines a reversibly sealable (i.e. unseal/seal) barrier to the container 1204 and the top (i.e. outer) edge thereof generally corresponds to the mouth 1206 of the container 1204.

Opposing lips (only one side visible) 1214 are provided to the mouth 1206 and these are moveable (once released, see later) to a “lips open” configuration, in which the lips form a guide for the receipt of a soiled item through the mouth 1206 and into the container 1204. First and second loops (only one visible) 1212 are further provided to the mouth 1206 and lips 1214. Each loop 1212 attaches between two points 1213a, 1213b of an outside wall of the container 1204, one of which is above and the other below the adhesive seal line 1210. It will be noted that the positioning of the second point of attachment 1213b is higher up the lips 1214 for the eighth device of FIG. 27b than for the seventh device of FIG. 27a. It will also be noted that the loop profile (i.e. the shape of the loop aperture) defined by the loops 1212 of each packaging and disposal device is arranged to enable ready receipt of a user's thumb/fingers.

The seventh and eighth packaging and disposal devices also comprise a tamper-proof seal 1217, which locates outer to the mouth 1206 and acts to hygienically seal off the interior of the container 1204 until the tamper-proof seal 1217 is broken. This action of breaking the tamper-proof seal 1217 acts to release the lips 1214, and also the loops 1212, which otherwise lie essentially flat against the container 1204. Locating upwards of the tamper-proof seal 1217 is an air capsule 1219 (e.g. bubble-like) one of wall of which corresponds to (FIG. 27a) or communicates with (FIG. 27b) the tear-off seal 1217. The air capsule 1219 provides a one-handed opening mechanism for the tamper-proof seal 1217 as is now described in more detail.

In a typical usage scenario, the seventh and eighth packaging and disposal devices are supplied with an item (e.g. a condom, tampon or sanitary towel, not shown) locating within the container 1204. To access that item for use thereof, the tamper-proof seal 1217 must be broken such that the item may then be accessed by opening the mouth 1206 (e.g. using the loops 1212 as mouth-opening means). To achieve that breaking of the tamper-proof seal 1217 the user applies pressure (e.g. by a finger and thumb pinching action) to the air capsule 1219, which causes rupturing of the tamper-proof seal 1217. The item may then be access and is then used, and such usage results in soiling thereof. The soiled item is then replaced into the container 1204, by opening the lips 1214 thereof by means of the loops (e.g. by a finger-thumb action) and placing the item through the mouth 1206. Once the soiled item has been received into the container 1204, the lips 1214 are closed and the container 1204 sealed off by means of the adhesive seal 1210. The soiled item may then be hygienically disposed of.

It will be appreciated that variations in, or modifications to, the embodiments described and illustrated may be made within the scope of the appended claims.

1-51. (canceled)

52. A disposal device for the disposal of a soiled item, the disposal device comprising a container substantially impervious to liquid having a mouth formed therein through which a soiled item may be received by the container, wherein the mouth is capable of manipulation by a single hand of a user between a mouth open position and a mouth closed position.

53. The disposal device of claim 52, further comprising mouth manipulation means attached to an outside wall of the container for manipulating the mouth between said mouth open position and said mouth closed position.

54. The disposal device of claim 53, wherein the mouth manipulation means comprises at least a first loop through which one or more fingers or a thumb of the user are receivable.

55. The disposal device of claim 54, wherein the mouth manipulation means further comprises a second loop through which one or more fingers, or a thumb are receivable, wherein the second loop is attached to an opposite outside wall of the container.

56. The disposal device of claim 55, wherein at least one of the loops may adopt both a first configuration in which the loop profile is closed and a second configuration in which the loop profile is open, wherein one or more folds are provided to the at least one loop such that the at least one loop readily adopts said second configuration in the absence of external pressure on the at least one loop.

57. The disposal device of claim 54, wherein the mouth manipulation means further comprises an area of adhesive located on an opposite outside wall of the container to which a person can adhere one or more finger or a thumb, to enable a person to adhere at least one finger or thumb to one wall and to insert at least one different finger or thumb through the loop located on said opposite wall, and to pull said fingers and/or thumb apart to open the mouth of the container with said single hand.

58. The disposal device of claim 52, wherein the mouth of the container is provided with lips for guiding the receipt of the soiled item into the mouth.

59. The disposal device of claim 58, wherein the mouth manipulation means are further arranged for manipulating the lips in either an open or closed position.

60. The disposal device of claim 59, wherein a first loop extends from a first lip and a second loop extends from a second lip.

61. The disposal device of claim 58, wherein the mouth manipulation means and the lips form an integral arrangement.

62. The disposal device of claim 52, wherein the mouth of the container is generally biased into a closed position and is opened upon manual application of a force.

63. The disposal device of claim 62, wherein the mouth comprises at least one flexing strip located at an entrance to the mouth on a wall of the container substantially parallel to the opening of the mouth, such that, in use, upon manual application of force to both ends of the flexing strip, the flexing strip is formed into an arc that causes the mouth of the container to open, wherein on release of the flexing strip, the flexing strip returns to its original shape and the mouth of the container returns to the closed position.

64. The disposal device of claim 63, wherein a further flexing strip is provided at the entrance to the mouth and arranged substantially parallel to the opening of the mouth on an opposite wall to the first flexing strip, such that, in use, upon manual of force to both ends of each flexing strip, they each become biased apart and form into respective arcs causing the mouth of the container to open, wherein on release of pressure to the flexing strips, the flexing strips return to their original shape and the mouth of the container returns to the closed position.

65. The disposal device of claim 52, wherein the container is provided with a seal such that a substantially liquid-tight seal to the container may be formed.

66. The disposal device of claim 65, wherein a layer of adhesive is provided on at least one internal boundary of the mouth, such that when the mouth is in the closed position, the mouth forms said seal.

67. The disposal device of claim 65, wherein the mouth comprises at least two strips provided on at least one of the opposing internal boundaries of the mouth, having sealing means to form the seal.

68. The disposal device of claim 67, wherein the sealing means comprise a tongue provided along a first strip and a corresponding groove provided along the second strip, wherein when the mouth is pressed into a closed position, the tongue and groove can engage together to form the seal.

69. The disposal device of claim 52 wherein the container is in the form of a sachet or pouch and the container is of a size and shape suitable to receive a tampon.

70. The disposal device of claim 52, further configured to act as a packaging device for the item in a pre-soiled state wherein the item is stored in the container prior to use, the disposal device further comprising a seal that seals off access to the mouth to protect the item in the pre-soiled state prior to use, such that the seal must be opened for the item to be removed and used.

71. A disposal device according to claim 70, wherein the seal is a tear-off seal.

72. A dispenser device arranged for storage of plural disposal devices as claimed in claim 52 adapted for dispensing one disposal device at a time therefrom.

73. A dispenser device as claimed in claim 72, wherein the dispenser device is arranged such that on dispensing said one disposal device the mouth manipulation means thereof is presented to the user for ready interaction with the user's fingers and/or thumb.

74. A dispenser device as claimed in claim 72, wherein the dispenser device is arranged for storage of said plural disposal devices in a stacking arrangement.

75. A dispenser device as claimed in claim 74, wherein in said stacking arrangement the uppermost disposal device of the stacking arrangement presents at least one manipulation means away therefrom for ready interaction with the user's fingers and/or thumb.

76. A dispenser device as claimed in claim 72, wherein the dispenser device is arranged for storage of said plural disposal devices in a rolled-up arrangement.

77. A dispenser device as claimed in claim 76, wherein in said rolled-up arrangement the lead disposal device of the rolled-up arrangement presents at least one manipulation means away therefrom for ready interaction with the user's fingers and/or thumb.

78. A method of using a disposal device for the disposal of a soiled item, the disposal device comprising a container substantially impervious to liquid having a mouth formed therein through which a soiled item may be received by the container, wherein the mouth is arranged to be capable of manipulation by a single hand of a user between a mouth open position and a mouth closed position, the method comprising the steps of:
opening the mouth of the container with a single hand;
placing a soiled item inside the container; and
closing the mouth of the container with the same single hand.

79. The method of claim 78, wherein the container of the disposal device prior to use contains the item in a pre-soiled state, the method further comprising the steps of removing the item and using the item prior to the step of placing the soiled item inside the container.

80. The method of claim 79, wherein the disposal device further comprises a seal sealing the item inside the container prior to use, the method further comprising the step of opening the seal prior to the step of removing the item.
